(19) United States
(12) Patent Application Publication
(10) Pub. No.: US 2011/0171328 A1
(43) Pub. Date: Jul. 14, 2011

(54) PHARMACEUTICAL COMPOSITION FOR TREATING DIABETES

(76) Inventors: Yingjun Zhou, Hunan (CN); Guangrao Zeng, Hunan (CN); Meixiang Jiang, Hunan (CN)

(21) Appl. No.: 13/000,361

(22) PCT Filed: Aug. 11, 2008
(86) PCT No.: PCT/CN2008/071950

§ 371 Date: Feb. 3, 2011

(30) Foreign Application Priority Data
Jun. 20, 2008 (CN) 200810031546.8

(51) Int. Cl. A61K 36/82; A61K 31/435; A61K 36/60; A61K 36/16; A61K 36/42; A61P 3/10

(52) U.S. Cl. 424/729; 514/345; 424/725; 424/752; 424/758

(57) ABSTRACT

The invention discloses a pharmaceutical composition for synergistically treating and preventing diabetic complication. The composition contains 1-deoxynojirimycin and fagomine, and the weight ration of the 1-deoxynojirimycin to the fagomine is 0.5-5:1.

### FIELD OF INVENTION

The invention relates to a pharmaceutical composition, in particular to a pharmaceutical composition for treating diabetes.

### BACKGROUND

1-deoxynojirimycin and fagomine are nitrogen-containing alkaloids contained in branches, leaves, bark or silkworm droppings of moraceae plants. The chemical name of 1-deoxynojirimycin is 3,4,5-trihydroxy-2-hydroxymethyl tetrahydropyridine. The compound has been commercialized and can be purchased from the market, there are many manufacturers providing the compound, such as Shanghai Winherb Medical S & T Development Co., Ltd.

There is no public sale of commodities of fagomine until now, and the literature has reported that Naoki Asano, et al. in Hokuriku University, Japan obtained fagomine through a method of extraction, repeated column chromatography and recrystallization (J. Carbohydrate Research, Vol. 253, pp, 235-245 (1994)).

Domestic and international researches and reports show that alkaloid extracts from moraceae plants have the roles of inhibiting the rise of blood glucose and improving glucose tolerance, wherein the 1-deoxynojirimycin can be combined with maltase, sucrase, lactase and other α-glycosidases in the small intestine, thereby avoiding further decomposition of disaccharides and further inhibiting the sharp rise in the postprandial blood glucose; and fagomine not only has the role of a glycosidase inhibitor, but also has the role of reducing the blood glucose by increasing the release of insulin.

Hypoglycemic chemicals including sulfonylureas such as glyburide, gliclazide and the like, biguanides such as metformin and the like, thiazolidinediones such as troglitazone, rosiglitazone and the like have been commercialized and applied clinically; and extracts of flavonoids from mulberry leaves, ginkgo biloba extract compound, bitter melon extract and tea polyphenol extract and the like are on sale in the market, such as by Hunan Geneham Biomedical Technology Ltd., Shaoguan Jumin Pharmaceutical Co., Ltd., and other medicine and extract manufactures.

### SUMMARY OF INVENTION

The invention aims at providing a pharmaceutical composition with better effect on reducing blood glucose.

The composition contains 1-deoxynojirimycin and fagomine, wherein the weight ratio of the 1-deoxynojirimycin to the fagomine is 0.5˜5:1.

Wherein, the preferential weight ratio of the 1-deoxynojirimycin to the fagomine is 2˜5:1.

The invention firstly researches the role of reducing the blood glucose of the composition of the 1-deoxynojirimycin and the fagomine with different mixing ratios, with the result that the two compounds have good synergistic hypoglycemic effect within a certain proportion range. According to the embodiments of the invention, the mixing ratio with better synergistic hypoglycemic effect is selected.

The invention further researches the synergistic hypoglycemic effect of the composition which is combined with other hypoglycemic compounds for use. On reducing the blood glucose value of rats with hyperglycemia after intragastric administration of sucrose, the joint application of the composition of the 1-deoxynojirimycin and the fagomine and other hypoglycemic substances can reduce blood glucose value more effectively than the other hypoglycemic substances or the combined application or the single application of the two.

The pharmaceutical composition containing 1-deoxynojirimycin and fagomine can be prepared into a variety of formulations, including tablets, capsules, granules, powder, dripping pills, oral liquid and the like. The preparation method can adopt the conventional excipients and the conventional methods,

The research results show that the pharmaceutical composition of present application has clear action mechanism and significant efficacy and can be used for treating and preventing diabetes and complications thereof.

### DESCRIPTION OF THE PREFERRED EMBODIMENTS

### Embodiment 1

Determination of α-D-glucosidase inhibitory activity of the combination compatibility of 1-deoxynojirimycin and fagomine.

1. Medicines: 1-deoxynojirimycin and fagomine, homemade.
2. Reagents: potassium dihydrogen phosphate, sodium hydroxide, sodium bicarbonate, para-nitrophenyl glucoside (PNPG), reduced glutathione and α-D-glucosidase sucrose.
3. Instrument: TU-1901 UV-Vis spectrophotometer.
4. Test method and results
(1) Preparation of potassium phosphate buffer solution: dissolve 0.68120 g of potassium dihydrogen phosphate in 25 mL of distilled water, obtain stock solution 1, dissolve 0.2 g of NaOH in 25 mL of the distilled water, obtain stock solution 2, suck the stock solution 1 (25 mL), add into the stock solution 2 (11.8 mL), shake up, dilute to 100 mL and obtain the potassium phosphate buffer solution.
(2) Na₂CO₃ stop solution: Na₂CO₃ 10.6 g→100 mL of distilled water (1 mol/L)
(3) PNPG: 17.47 mg→2 mL of distilled water (0.029 mol/L)
(4) Reduced glutathione: 2.10 mg→2 mL of distilled water (1.05 mg/mL)
(5) α-D-glucosidase sucrose: 0.5 mg→1 mL of distilled water (0.5 mg/mL)
(6) 1-deoxynojirimycin stock solution: 3.24 mg→1 mL of distilled water (20 μmol/mL)
(7) Fagomine stock solution: 3.00 mg→1 mL of distilled water (20 μmol/mL)
(8) Preparation of glycosidase inhibitor sample solution:

Take 250 μL of the 1-deoxynojirimycin stock solution, dilute to 1 mL with the distilled water, and obtain the stock solution 1.

Take 250 μL of the fagomine stock solution, dilute to 1 mL with the distilled water, and obtain the stock solution 2.

The compatibility of the stock solution 1 and the stock solution 2 is carried out according to the proportion in Table 2.

(9) Determination reaction of α-D-glucosidase inhibitory activity

Add the reagents according to the sequence in Table 1 for reaction, determine the absorbance value and calculate the α-glycosidase inhibition ratio of a sample according to the following formula:

$$α \text{-} glycosidase inhibition ratio \% = \frac{\begin{matrix}Absorbance of sample \\ tube \text{-} absorbance \\ of blank tube\end{matrix}}{\begin{matrix}Absorbance of standard \\ tube \text{-} absorbance \\ of blank tube\end{matrix}} \times 100 \%$$

TABLE 1
Design of Determination Test of α-glycosidase Inhibition Ratio

| | | Standard | |
|---|---|---|---|
| Reagent (μL) | Blank centrifuge tube | tube | Sample tube |
| Potassium phosphate | 2500 | 2500 | 2500 |
| buffer solution | | | |
| α-D-glucosidase | 15 | 15 | 15 |
| Reduced glutathione | 15 | 15 | 15 |
| Distilled water | 45 | 30 | |
| Glycosidase inhibitor | | | 30 |

Constant temperature for 10 min in water bath cauldron at 37° C.

| | | |
|---|---|---|
| PNPG | 15 | 15 |

Constant temperature for 10 min in water bath cauldron at 37° C.

| | | | |
|---|---|---|---|
| Na₂CO₃ stop solution | 1425 | 1425 | 1425 |

Colorimetry at UV 400 nm

### (10) Results: see Table 2.

TABLE 2
Test of α-glycosidase Inhibition Ratio of 1-deoxynojirimycin and
Fagomine in Different Mixing Ratios

| | | | | | | | |
|---|---|---|---|---|---|---|---|
| DNJ:Fag | 1:0 | 5:1 | 2:1 | 1:1 | 1:2 | 1:5 | 0:1 |
| mixing ratio | | | | | | | |
| DNJ:Fag sample | 30:0 | 25:5 | 20:10 | 15:15 | 10:20 | 5:25 | 0:30 |
| solution volume | | | | | | | |
| α-glycosidase | 44% | 59% | 54% | 48% | 42% | 33% | 21% |
| inhibition ratio % | | | | | | | |

Note:
In the table, 1-deoxynojirimycin is abbreviated as DNJ and fagomine is abbreviated as Fag.

5. Conclusion: the test results show that the fagomine and the 1-deoxynojirimycin have a synergistic α-glycosidase inhibition role (the total amount of the 1-deoxynojirimycin and the fagomine is constant), when the mixing ratio of the 1-deoxynojirimycin to the fagomine is 0.5-5:1, the α-glycosidase can be better inhibited, wherein when the mixing ratio is 2-5:1, the effect is plainly better than the effect of the single use of the 1-deoxynojirimycin in the same concentration.

### Embodiment 2

Impacts of the composition in different doses on blood glucose and serum insulin of mice with spontaneous hyperglycemia

1, Medicines: 1-deoxynojirimycin and fagomine, prepared by the inventor. The two are prepared into the required concentrations with distilled water before the test (the sum of the concentrations of the 1-deoxynojirimycin and the fagomine in the composition is the same as the concentration of the fagomine or the 1-deoxynojirimycin which is prepared alone). Glibenclamide is obtained from Hunan Dongting Pharmaceutical Co., Ltd.
2. Animals: NOD mice with the weight of 25-35 g, provided by Experimental Animal Center, Peking University Health Science Center. Determine the blood glucose before the test and select the animals with the blood glucose value of above 11.1 mmol/L for carrying out the test.
3. Determination instrument: Yicheng blood glucose tester, developed by Beijing Yicheng Bioelectronic Technology Co., Ltd.
4. Main reagents: ¹²⁵I-insulin radioimmunoassay kit, produced by Department of Isotopes, China Institute of Atomic Energy.
5. Test method and results
(1) Impacts on the blood glucose of the mice: determine the blood glucose of the NOD mice after fasting for 6 h, select 70 animals with blood glucose above 11.1 mmol/L, randomly divide into seven groups, respectively carry out intragastric administration and give the following medicines: i) control group (distilled water); ii) glibenclamide group (50 mg/kg); and iii)-v) composition in different mixing ratios (20 mg/kg, with different medicine proportions and constant total amount). Carry out the continuous administration for 14 days, fast for 6 h before the administration at the 14^{th} day, draw blood from the animals 1 h after the administration, use the Yicheng blood glucose tester to determine the blood glucose value of the animals, and determine the serum insulin by radioimmunoassay. The results are shown in Tables 3-4.

TABLE 3
Impacts of Pharmaceutical Composition on Blood Glucose of Mice
with Spontaneous Hyperglycemia (X ± SD)

| | | | Blood glucose | |
|---|---|---|---|---|
| | | Number | before | Blood glucose after |
| | Dose | of | administration | administration |
| Group | (mg/kg) | animals | (mmol/L) | (mmol/L) |
| Model control | — | 10 | 13.64 ± 2.08 | 13.97 ± 1.86 |
| group | | | | |
| Glibenclamide | 50 | 10 | 13.26 ± 2.26 | 10.57 ± 2.17** |
| DNJ | 20 | 10 | 14.20 ± 1.33 | 10.60 ± 2.13** |
| Fagomine | 20 | 10 | 13.49 ± 1.65 | 12.24 ± 1.62** |
| Composition | 20 | 10 | 14.14 ± 1.70 | 9.75 ± 1.53** |
| Composition | 20 | 10 | 13.58 ± 1.73 | 10.04 ± 1.44** |
| Composition | 20 | 10 | 13.92 ± 2.07 | 10.39 ± 1.43** |

Note:
1. Compare with the model control group: *P < 0.05, **P < 0.01 (t-test).
2. In the table, 1-deoxynojirimycin is abbreviated as DNJ and fagomine is abbreviated as Fag.

TABLE 4
Impacts of Pharmaceutical Composition on Serum Insulin of Mice with
Spontaneous Hyperglycemia (X ± SD)

| | Dose | Number of | Serum insulin |
|---|---|---|---|
| Group | (mg/kg) | animals | (MIU/L) |
| Model control group | — | 10 | 14.84 ± 2.83 |
| Glibenclamide | 50 | 10 | 22.57 ± 3.05** |
| 1-deoxynojirimycin | 20 | 10 | 16.14 ± 2.63 |
| Fagomine | 20 | 10 | 21.78 ± 3.34** |
| Composition (DNJ:Fag = 5:1) | 20 | 10 | 17.25 ± 2.15* |
| Composition (DNJ:Fag = 2:1) | 20 | 10 | 19.83 ± 2.65** |
| Composition (DNJ:Fag = 1:2) | 20 | 10 | 21.03 ± 3.22** |

Note:
1. Compare with the model control group: *P < 0.05, **P < 0.01 (t-test).
2. In the table, 1-deoxynojirimycin is abbreviated as DNJ and fagomine is abbreviated as Fag.

6. Conclusion: The single application of 1-deoxynojirimycin can significantly reduce the blood glucose of the NOD mice and has better effect than the single application of fagomine for reducing the blood glucose; and the single application of the 1-deoxynojirimycin has little effect on the serum insulin of the NOD mice, and the single application of the fagomine can significantly improve the serum insulin level.

When the mixing ratio of the 1-deoxynojirimycin to the fagomine is 2-5:1, the composition can improve the serum insulin level of the NOD mice and significantly reduce the blood glucose of the NOD mice, and the hypoglycemic effect is plainly better than the single application of 1-deoxynojirimycin or of fagomine with the same concentration, showing that the combined application of the two has synergistic effect.

In addition, according to the results of the efficacy test, the effective dose of the composition of the 1-deoxynojirimycin and the fagomine approximately equals to 120 mg of the composition per person per day, with about 40 mg each time; calculated by the preferential proportion of 2-5:1.

Each administration at least contains more than 25 mg of the 1-deoxynojirimycin. From the aspects of facilitating the administration to patients and reducing the administration dose, tablets or capsules are adopted, and each administration contains 5 tablets or 5 capsules at most (the total amount is calculated as 2500 mg at most), so that the preferential content of the 1-deoxynojirimycin in the pharmaceutical composition is larger than 1%.

### Embodiment 3

Joint application test with other hypoglycemic substances

1. Medicines: 1-deoxynojirimycin-fagomine mixture (2:1), homemade. The mixture is prepared to the required concentration with distilled water before the test. Glucobay is obtained from Bayer Healthcare Co., Ltd. Catechin, ginkgo biloba extract and flavonoids from mulberry leaves were obtained from Hunan Geneham Biomedical Technology Ltd.
2. Animals: SD rats with the weight of 180-220 g, half male and half female, provided by Hunan Institute of Pharmaceutical Industry Research. Modeling method: carry out small dose intravenous injection of 25 mg/kg of streptozotocin on the rats after fasting for 24 h, once a week, simultaneously heat feed for feeding, induce H type diabetes, determine the blood glucose 1 week after the second injection, and select the animals with the fasting blood glucose of above 7.8 mmol/L for carrying out the test.
3. Reagents: streptozotocin, product of Sigma Company, USA. Use citric acid buffer solution (PH=4.0) before modeling, and prepare at low temperature; sucrose, analytically pure.
4. Instrument: SURESTEP PLUS blood glucose monitoring meter.
5. Test method and results: lead the SD rats to fast without water deprivation for 24 h, carry out intravenous injection of 25 mg/kg of streptozotocin, once a week, simultaneously heat feed for feeding, induce II type diabetes, determine the blood glucose 1 week after the second injection, and select 90 rats with the fasting blood glucose of above 7.8 mmol/L for carrying out the test. Randomly divide into 9 groups (n=10), wherein the first group is the control group with isometric distilled water for intragastric administration, the second group is the glucobay group, and other groups are the 1-deoxynojirimycin-fagomine mixture (the proportion of the mixture is 2:1, represented as DF in the table) and compositions of other different hypoglycemic substances; and carry out intragastric administration according to the above dose, give the test medicines continuously for 14 days, fast for 6 h before the administration at 14^{th} day, carry out the administration according to the above dose, simultaneously carry out the intragastric administration with 5.0 g/kg of sucrose, and determine the blood glucose of the rats before sucrose administration and 1.0 h after the sucrose administration, wherein the temperature in a laboratory is 20° C.-25° C. The test results are shown in Table 5.

TABLE 5
Impacts of Composition on Blood Glucose Value of Rats with
Streptozotocin-induced Hyperglycemia after Intragastric Administration
with Sucrose

| | | Blood glucose value of rats | |
|---|---|---|---|
| | Medicine | with hyperglycemia (mmol/L) | |
| | dose | Before | |
| Group | (mg/kg) | administration | After administration |
| Model control group | Isometric | 8.45 ± 0.82 | 15.56 ± 2.54 |
| | distilled | | |
| | water | | |
| Glucobay group | 10.0 | 8.24 ± 0.93 | 12.21 ± 1.84** |
| DF:catechin (1:2) | 60 | 8.97 ± 1.01 | 12.47 ± 2.03** |
| Catechin | 60 | 9.17 ± 0.89 | 14.10 ± 1.50 |
| DF:ginkgo biloba | 60 | 8.31 ± 0.94 | 12.54 ± 1.61** |
| extract (1:2) | | | |
| Ginkgo biloba extract | 60 | 8.56 ± 1.11 | 13.82 ± 1.72 |
| DF:flavonoids from | 60 | 7.96 ± 1.07 | 12.04 ± 1.91** |
| mulberry leaves | | | |
| Flavonoids from | 60 | 8.72 ± 1.22 | 13.65 ± 2.12* |
| mulberry leaves | | | |
| DF | 20 | 8.37 ± 1.05 | 12.88 ± 1.55** |

Note:
Compare with the model control group:
*P < 0.05,
**P < 0.01.

6. Conclusion: The composition of the 1-deoxynojirimycin and the fagomine and other hypoglycemic substances can more effectively reduce the blood sugar value of the rats with the hyperglycemia after the intragastric administration of the sucrose in comparison with the combined and single applications of other hypoglycemic substances and DF.

1. A pharmaceutical composition for treating diabetes, wherein the composition contains 1-deoxynojirimycin and fagomine, and the weight ratio of the 1-deoxynojirimycin to the fagomine is 0.5-5:1.

2. The pharmaceutical composition of claim 1, wherein, the weight percent of the 1-deoxynojirimycin is more than 1%.

3. The pharmaceutical composition of claim 1, wherein, the preferential weight ratio of the 1-deoxynojirimycin to the fagomine is 2-5:1.

4.-5. (canceled)

6. The pharmaceutical composition of claim 1, wherein the composition contain other compounds or extracts with hypoglycemic activity.

7. The pharmaceutical composition of claim 2, wherein the composition contain other compounds or extracts with hypoglycemic activity.

8. The pharmaceutical composition of claim 3, wherein the composition contain other compounds or extracts with hypoglycemic activity.

9. The pharmaceutical composition for treating diabetes of claim 6, wherein other compounds with the hypoglycemic activity are selected from chemicals of sulfonylureas, biguanides and thiazolidinediones; the extracts can be selected from mulberry leaf flavonoids separated and purified from moraceae plants, ginkgo biloba extract, bitter melon extract and tea polyphenol extract.

10. The pharmaceutical composition for treating diabetes of claim 7, wherein other compounds with the hypoglycemic activity are selected from chemicals of sulfonylureas, biguanides and thiazolidinediones; the extracts can be selected from mulberry leaf flavonoids separated and purified from moraceae plants, ginkgo biloba extract, bitter melon extract and tea polyphenol extract.

11. The pharmaceutical composition for treating diabetes of claim 8, wherein other compounds with the hypoglycemic activity are selected from chemicals of sulfonylureas, biguanides and thiazolidinediones; the extracts can be selected from mulberry leaf flavonoids separated and purified from moraceae plants, ginkgo biloba extract, bitter melon extract and tea polyphenol extract.
